(19) United States
(12) Patent Application Publication
(10) Pub. No.: US 2017/0029879 A1
(43) Pub. Date: Feb. 2, 2017

(54) IMPROVED SENSITIVITY FOR MOLECULAR DETECTION OF MICROBES IN THE BLOODSTREAM

(71) Applicant: Ibis Biosciences, Inc., Carlsbad, CA (US)

(72) Inventor: Rangarajan SAMPATH, San Diego, CA (US)

(21) Appl. No.: 15/117,116

(22) PCT Filed: Feb. 6, 2015
(86) PCT No.: PCT/US2015/014768

§ 371 Date: Aug. 5, 2016

Related U.S. Application Data
(60) Provisional application No. 61/937,264, filed on Feb. 7, 2014.

(51) Int. Cl. C12Q 1/68; H01J 49/00; H01J 49/16

(52) U.S. Cl. CPC C12Q 1/6846; H01J 49/165; H01J 49/0031

(57) ABSTRACT

The present disclosure relates to systems and methods for detection of microbes in the bloodstream. In particular, the present invention relates to systems and methods for detection of bacteria and yeast in the bloodstream that are indicative of sepsis.

### FIELD OF INVENTION

The present disclosure relates to systems and methods for detection of microbes in the bloodstream. In particular, the present invention relates to systems and methods for detection of bacteria and yeast in the bloodstream that are indicative of sepsis.

### BACKGROUND

Although molecular tests have been available for decades, there is no widely used molecular method to rapidly identify the organisms causing bloodstream infections and bacteremia directly from the blood. In the most serious cases—those of sepsis and septic shock—the risk of mortality increases by the hour if appropriate antimicrobial therapy is delayed (1). The long delays associated with culture methods for detection and identification of organisms in blood force physicians to empirically treat patients with multiple broad-spectrum antimicrobial agents rather than waiting for more specific microbiological data. This is not ideal because of the toxicities of broad-spectrum agents, the fact that the empiric antimicrobial therapy might not be optimal for the infection, the high costs associated with increased hospitalization time, and the impact on antimicrobial stewardship (1-5).

Direct molecular identification of infecting microbes in blood would mitigate these issues. PCR and mass spectrometry methods used on positive blood cultures to identify microbes have been reported to decrease the time to an answer, but this strategy is still far from ideal as it depends on cultures to grow. More importantly, blood cultures are negative in more than 50% of the cases where true bacterial or candida infections are believed to exist (3, 6, 7). Some of these apparent false-negatives result from a lack of bacteria in any one sample; however, often the bacteria are present in such samples are rendered non-viable (and hence unculturable) by concurrent antibiotic treatment (10). Molecular methods have an inherent advantage in detecting such cryptic infections, as they do not rely on viability.

A rapid and sensitive molecular method capable of detecting a broad range of microbes directly in blood specimens would have a game-changing impact on the management of patients with suspected infections. However, there are significant challenges involved in molecular detection and identification of microbes present in low quantities in blood. First, a relatively large volume of blood must be analyzed to provide a reasonable sampling of the blood compartment where the distribution of the microbe may not be homogeneous (8-10). Second, diverse microbes must be efficiently lysed in a dense, complex matrix of cellular material. Third, high quantities of human genomic DNA from white blood cells must either be co-purified with microbial DNA or separated without losing the microbial DNA. Finally, amplification of target DNA and signal capture (by sequencing, probe capture, mass spectrometry, or another method) must be robust. This can be very challenging if genomic DNA is co-purified with targeted microbial DNA. (11-13). A number of solutions have been proposed to solve these problems (14-19), but to date none have achieved the desired sensitivity (20). It should be noted that many of these methods do appear to capture a significantly higher number of total positives than culture, and the “extra” detections are often correlated with clinical indications of bloodstream infection—however, an inability to capture all positives detected by culture has been seen as an Achilles' heel.

Thus, a need remains for efficient and sensitive methods of detecting blood borne infections.

### SUMMARY

The present disclosure relates to systems and methods for detection of microbes in the bloodstream. In particular, the present invention relates to systems and methods for detection of bacteria and yeast in the bloodstream that are indicative of sepsis.

Embodiments of the present invention provide a method of detecting a microbe in a sample (e.g., blood sample), comprising a) lysing a blood sample (e.g., whole blood sample) (e.g., a sample of 1 to 10 ml (e.g., 2 to 8 ml, 4 to 6 ml, or 5 ml) of whole blood) from a subject in a lysis buffer comprising yttria-stabilized zirconium oxide beads (e.g., using a large-volume bead mill homogenizer); b) processing the supernatant fractions from the lysis (e.g., using an automated DNA extraction system that uses pre-filled disposable cartridges containing DNA-free reagents and silica-coated magnetic particles); and c) performing a PCR reaction on the eluate of the processing (e.g., using a plurality of PCR primer pairs, wherein each of the PCR primer pairs hybridizes to a conserved genomic sequence of a microbe). In some embodiments, the method further comprises the step of performing electrospray ionization mass spectrometry (ESI-MS) on amplicons of said PCR reaction, wherein the ESI-MS determines the presence or absence of the microbe in the sample, the identity of the microbe(s) in the sample, and/or the presence or absence of antibiotic resistance genes in the microbe in the sample. In some embodiments, the PCR reaction utilizes primer pairs at a concentration of 200 to 1500 μM (e.g., 500 to 100 μM, 600 to 800 μM or 750 μM) and polymerase at a concentration of 0.5 to 5 units (e.g., 1 to 4 units, 1.5 to 3 units, or 2.2 units) per reaction. In some embodiments, the PCR reaction further comprises a plurality of target specific primers that hybridize to antibiotic resistant elements of microbial DNA. In some embodiments, the microbes are bacteria (e.g., K. pneumonia, E. faecium, or S. aureus) or yeast (e.g., C. albicans). In some embodiments, the bacteria comprise one or more antibiotic resistance genes. In some embodiments, the PCR reaction is configured to amplify microbial DNA in a sample comprising up to 12 μg of human DNA per reaction. In some embodiments, the lysis buffer comprises 3 g of 0.2-mm yttria-stabilized zirconium oxide beads. In some embodiments, the method detects microbes with a limit of detection of 50 (e.g., 20, 19, 18, 17, 16, 15, 14, 13, 12, 11, 10, 9, 8, 7, 6, 5, 4, 3, or 2) CFU/ML or less. In some embodiments, the presence of the microbe in the sample is indicative of a diagnosis of sepsis, septic shock, pneumonia, or a blood stream infection in the subject. In some embodiments, the method further comprises the step of determining a treatment course of action based on the diagnosis (e.g., administration of an antibiotic or anti-fungal agent to the subject). In some embodiments, the present invention further provides the step of administering the treatment to the subject. In some embodiments, the treatment is an antibiotic directed towards microbes identified in the sample. In some embodiments, the antibiotic is selected based on the antibiotic resistance genes identified in the microbes (e.g., an antibiotic that the microbe is not resistant to). In some embodiments, the treatment is a narrow spectrum antibiotic targeted specifically to the microbes identified in the sample.

In further embodiments, the present invention provides the use of the methods described herein to diagnose or monitor sepsis, septic shock, pneumonia, or a blood stream infection in a subject.

In yet other embodiments, the present invention provides a kit, comprising reagents useful, necessary, or sufficient for detecting a microbe in a blood sample (e.g., whole blood sample), selected from, for example, a lysis buffer comprising yttria-stabilized zirconium oxide beads, a plurality of PCR primer pairs, wherein each of the PCR primer pairs hybridizes to a conserved genomic sequence of a microbe, a plurality of target specific primers that hybridize to antibiotic resistant elements of microbial DNA, and a DNA polymerase.

In still further embodiments, the present invention provides a system, comprising: a lysis buffer comprising yttria-stabilized zirconium oxide beads, a plurality of PCR primer pairs, wherein each of the PCR primer pairs hybridizes to a conserved genomic sequence of a microbe, optionally a plurality of target specific primers that hybridize to antibiotic resistant elements of microbial DNA, a DNA polymerase; a homogenizer (e.g., large-volume bead mill homogenizer); a DNA extraction system (e.g., an automated DNA extraction system that uses pre-filled disposable cartridges containing DNA-free reagents and silica-coated magnetic particles); and optionally an ESI-MS instrument.

The present invention also provides a reaction mixture comprising a plurality of PCR primer pairs, wherein each of the PCR primer pairs hybridizes to a conserved genomic sequence of a microbe hybridized to a microbial nucleic acid sample isolated using any of the above described method

Additional embodiments are described herein.

### BRIEF DESCRIPTION OF THE DRAWINGS

These and other features, aspects, and advantages of the present technology will become better understood with regard to the following drawings:

FIG. 1 shows work flow and timing of the steps in sample preparation and PCR/ESI-MS analysis of whole blood specimens.

FIG. 2 shows a frequency distribution plot for white blood cell counts obtained from patients.

FIG. 3 shows that microbial and human DNA load define the functional limits of 16S sequence analysis of clinical specimens.

FIG. 4 shows quantitative bacterial loads in whole blood determined by various methods. Legend Symbols: Q=Inter-Quartile Range, I=Range, S=+/−1 standard deviation, |=Cutoff, diamond=Median.

FIG. 5 shows spectra from representative PCR reactions. Spectra for primer pairs 348 (left column) and 349 (right column) are reported for sample 1083 (Serratia marcescens detected in replicates 1 and 2, first and second rows) and for sample 933 (Acinetobacter baumannii detected in replicates 1 and 2, third and fourth rows).

### DETAILED DESCRIPTION

The present disclosure relates to systems and methods for detection of microbes in the bloodstream. In particular, the present invention relates to systems and methods for detection of bacteria and yeast in the bloodstream that are indicative of sepsis.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present technology.

As used herein, the term “about” means encompassing plus or minus 10%. For example, about 90% refers to a range encompassing between 81% and 99% nucleotides. As used herein, the term “about” is synonymous with the term approximately.

As used herein, the term “amplicon” or “bioagent identifying amplicon” refers to a nucleic acid generated using the primer pairs described herein. The amplicon is typically double stranded DNA; however, it may be RNA and/or DNA:RNA. In some embodiments, the amplicon comprises DNA complementary to herpesvirus DNA, or cDNA. In some embodiments, the amplicon comprises sequences of conserved regions/primer pairs and intervening variable region. As such, the base composition of any given amplicon may include the primer pair, the complement of the primer pair, the conserved regions and the variable region from the bioagent that was amplified to generate the amplicon. One skilled in the art understands that the incorporation of the designed primer pair sequences into an amplicon may replace the native sequences at the primer binding site, and complement thereof. In certain embodiments, after amplification of the target region using the primers the resultant amplicons having the primer sequences are used to generate the molecular mass data. Generally, the amplicon further comprises a length that is compatible with mass spectrometry analysis. Bioagent identifying amplicons generate base compositions that are preferably unique to the identity of a bioagent.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

The term “detect”, “detecting” or “detection” refers to an act of determining the existence or presence of one or more targets (e.g., microorganism nucleic acids, amplicons, etc.) in a sample.

As used herein, the term “etiology” refers to the causes or origins, of diseases or abnormal physiological conditions.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, the term “molecular mass” refers to the mass of a compound as determined using mass spectrometry, for example, ESI-MS. Herein, the compound is preferably a nucleic acid. In some embodiments, the nucleic acid is a double stranded nucleic acid (e.g., a double stranded DNA nucleic acid). In some embodiments, the nucleic acid is an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

### Embodiments of the Technology

Embodiments of the present disclosure provide systems and methods for the rapid identification of bacteria and fungi directly from the blood of patients with suspected bloodstream infections. Such methods aid in diagnosis and guide treatment decisions. Development of an automated, rapid, and sensitive molecular technology capable of detecting the diverse agents of such infections at low titers has been challenging in part due to the high background of genomic DNA in blood. PCR followed by electrospray ionization mass spectrometry (PCR/ESI-MS) allows rapid and accurate identification of microorganisms, but with sensitivity of about 50% as compared to culture when using 1-ml whole blood specimens. The present disclosure describes an integrated specimen preparation technology that substantially improves sensitivity of a PCR/ESI-MS analysis. An efficient lysis method and automated DNA purification system were designed for processing of, for example, 5-ml of whole blood. In addition, PCR amplification formulations were implemented to tolerate high levels of human DNA. Analysis of samples collected from 331 patients with suspected bloodstream infections resulted in 35 PCR/ESI-MS positive specimens (10.6%) compared to 18 positive by culture (5.4%). PCR/ESI-MS was 83% sensitive and 94% specific as compared to culture. Replicate PCR/ESI-MS testing from a second aliquot of the PCR/ESI-MS positive, culture negative specimens corroborated the initial findings in most cases, resulting in an increased sensitivity (91%) and specificity (99%) when these confirmed detections were considered true positives. The integrated solution described provides rapid detection and identification of organisms responsible for bloodstream infections.

In order for molecular assays to be optimally useful for diagnosis of patients with suspected systemic infections, the assay should 1) accurately and sensitively identify bacterial and candida species present in blood, 2) provide results within hours, 3) detect the most important genetic mediators of antimicrobial drug resistance, and 4) be carried out with a work flow and throughput suitable for a hospital laboratory. To meet these objectives, experiments described herein resulted in the development of a PCR/ESI-MS hardware platform with clinically necessary sensitivity that facilitates a robust implementation of workflow. This was achieved by extracting nucleic acids from a 5-mL volume of blood, developing an automated specimen preparation technology to accommodate this volume, and optimizing the entire remaining system to be tolerant to the high levels of human DNA arising from the human white blood cells. Unlike protocols that separate human white blood cells from the bacteria or those that separate the bacterial DNA from the human DNA after lysis, the procedure retains all potential compartments of bacterial DNA signals, including cell-associated and free bacteria and free bacterial DNA, and avoids introduction of steps that complicate the workflow and increase costs.

This improved approach resulted in PCR/ESI-MS detecting twice as many positive samples (10.6%) as culture (5.4%) in the 331 samples analyzed from patients suspected of a blood infection. This is consistent with the well-established observation that approximately half the truly infected patients are not positive by culture (3, 6, 7), often because samples were taken after patients began antimicrobial drug treatment. The analytical sensitivity for uninfected blood spiked with bacteria was improved about 5 fold compared to the previously reported 1-mL sample preparation method(28, 29). More importantly, the higher volume sample preparation method integrated with the other sensitivity improvements increased the detection rate for specimens that were blood-culture positive from about 50% for the low-blood volume PCR-ESI-MS assay to 83% for the high-volume protocol.

A highly sensitive molecular test that detects a broad range of bacteria and fungi presents challenges for validation. Although PCR/ESI-MS-positive results can be compared to positive culture results, there is no good way to corroborate a PCR/ESI-MS positive result when cultures are negative, other than showing that PCR/ESI-MS gives the same result when testing additional specimens from the same patient. When replicate testing was performed, 91% of PCR/ESI-MS results were corroborated. Broad amplification followed by sequencing failed to provide a sufficiently sensitive comparator method for bloodstream infections because it is difficult to amplify and sequence the small amount of targeted bacterial DNA in the overwhelming background of human DNA.

There is a growing body of evidence that rapid and accurate identification of the microbes causing bloodstream infections can provide significant clinical and economic value. Use of molecular methods to identify culture-isolated organisms decreases the overall time to identification, resulting in improved patient outcomes and significantly decreasing hospital costs (55-58). This is a strong step forward, but requires time for culture and is not useful when cultures from truly infected patients are negative (59, 60). The direct analysis methods described herein both increases the number of patients who benefit from the information and further decreases the time to result by avoiding the lag time associated with growing cultures.

Accordingly, embodiments of the present disclosure provide a sensitive and efficient method of detecting microbes in blood samples (e.g., whole blood samples). In some embodiments, the method comprises the steps of: a) lysing a blood sample (e.g., whole blood sample) (e.g., a sample of 1 to 10 ml (e.g., 2 to 8 ml, 4 to 6 ml, or 5 ml) of whole blood) from a subject in a lysis buffer comprising yttria-stabilized zirconium oxide beads (e.g., using a large-volume bead mill homogenizer); b) processing the supernatant fractions from the lysis (e.g., using an automated DNA extraction system that uses pre-filled disposable cartridges containing DNA-free reagents and silica-coated magnetic particles); and c) performing a PCR reaction on the eluate of the processing (e.g., using a plurality of PCR primer pairs, wherein each of the PCR primer pairs hybridizes to a conserved genomic sequence of a microbe); and performing electrospray ionization mass spectrometry (ESI-MS) on the amplicons. In some embodiments, the PCR reaction utilizes primer pairs at a concentration of 200 to 1500 μM (e.g., 500 to 100 μM, 600 to 800 μM or 750 μM) and polymerase at a concentration of 0.5 to 5 units (e.g., 1 to 4 units, 1.5 to 3 units, or 2.2 units) per reaction. In some embodiments, the PCR reaction is optimized to detect microbe DNA in the presence of human genomic DNA (e.g., up to 5, 10, 12, or more μg of human DNA per reaction). In some embodiments, the method detects microbes with a limit of detection of 50 (e.g., 20, 19, 18, 17, 16, 15, 14, 13, 12, 11, 10, 9, 8, 7, 6, 5, 4, 3, or 2) CFU/ML or less.

In some embodiments, the compositions, systems and method described herein are configured to detect microbial nucleic acids at a low LOD in blood samples in a large volume of unpurified whole blood (e.g., at least 5 ml). the combination of lysis methods and optimized PCR reactions provides sensitive and specific detection of microbes in the presence of contaminating genomic DNA. The compositions, systems, and methods are able to identify the species of microbe and the presence or absence of antibiotic resistance genes in a high % of positive samples (e.g., at least 80%, at least 85%, at least 90%, at least 95%, at least 99%, or all).

The present disclosure is not limited to the detection of a particular microorganism. In some embodiments, the present disclosure provides systems and methods for detected microorganism (e.g., bacteria and yeast) responsible for blood borne infections (e.g., sepsis).

In some embodiments, the present invention provides zirconia/yttria compositions (e.g. surfaces, beads, substrates, devices, etc.). In some embodiments, the present invention provides zirconia/yttria compositions for the purification, isolation, preparation, and/or analysis of nucleic acids (e.g. total DNA and RNA). In some embodiments, compositions of the present invention comprise yttrium and/or zirconium. In some embodiments zirconia/yttria compositions comprise ZrO₂ (zirconium dioxide) and/or Y₂O₃ (yttrium oxide). In some embodiments, the present invention provides yittria stabilized zirconia (YSZ). In some embodiments, the present invention provides zirconium-oxide based ceramic. In some embodiments, the crystal structure of zirconium oxide is made stable at room temperature by an addition of yttrium oxide. In some embodiments, compositions comprise 95% ZrO₂ and 5% Y₂O₃. In some embodiments, compositions comprise approximately 95% ZrO₂ and 5% Y₂O₃. In some embodiments, compositions comprise at least 70% ZrO₂ (e.g. 70% ZrO₂, 75% ZrO₂, 80% ZrO₂, 85% ZrO₂, 90% ZrO₂, 95% ZrO₂, 99% ZrO₂, >99% ZrO₂, etc.). In some embodiments, compositions comprise greater than about 70% ZrO₂ (e.g. approximately 71% ZrO₂, approximately 75% approximately 80% ZrO₂, approximately 85% ZrO₂, approximately 90% ZrO₂, approximately 95% ZrO₂, approximately 99% ZrO₂, >99% ZrO₂, etc.). In some embodiments, compositions comprise 30% or less Y₂O₃ (e.g. 30% Y₂O₃, 25% Y₂O₃, 20% Y₂O₃, 15% Y₂O₃, 10% Y₂O₃, 5% Y₂O₃, 1% Y₂O₃, <1% Y₂O₃, etc.). In some embodiments, compositions comprise less than about 30% Y₂O₃ (e.g. approximately 29% Y₂O₃, approximately 25% Y₂O₃, approximately 20% Y₂O₃, approximately 15% Y₂O₃, approximately 10% Y₂O₃, approximately 5% Y₂O₃, approximately 1% Y₂O₃, <1% Y₂O₃, etc.). In some embodiments, compositions of the present invention may comprise additional compounds and/or compositions in addition to zirconia/yttria. In some embodiments, compositions may comprise calcia-stabilized zirconia, magnesia-stabilized zirconia, ceria-stabilized zirconia or alumina-stabilized zirconia. In some embodiments, compositions may contain an amount of impurities acceptable to those of skill in the art. In some embodiments, compositions are devoid of silica. In some embodiments, compositions are devoid of a substantial amount of silica. In some embodiments, the present invention provides beads of a suitable size for molecular biology purposes as would be understood by one of skill in the art. In some embodiments, the present invention provides beads with a mean diameter of greater than 1 μm (e.g. about 2 μm, about 5 μm, about 10 μm, about 20 μm, about 50 μm, about 100 μm, about 200 μm, about 500 μm, about 1 mm, about 2 mm, about 5 mm, about 1 cm, >1 cm, etc.).

In some embodiments, the present invention provides buffers and reagents for use with zirconia/yttria compositions (e.g. for storage, use in purification of nucleic acid, charging, cleaning, etc.). In some embodiments, the present invention provides an appropriate salts (e.g. NaCl, KOH, MgCl₂, etc.) and salt concentration (e.g. high salt, low salt, 1 mM, 2 mM, 5 mM, 10 mM, 20 mM, 50 mM, 100 mM, 200 mM, 500 mM, 1 M, etc.) for use with zirconia/yttria compositions. In some embodiments, buffers for use with zirconia/yttria compositions may include, but are not limited to H₃PO₄/NaH₂PO₄, Glycine, Citric acid, Acetic acid, Citric acid, MES, Cacodylic acid, H₂CO₃/NaHCO₃, Citric acid, Bis-Tris, ADA, Bis-Tris Propane, PIPES, ACES, Imidazole, BES, MOPS, NaH₂PO₄/Na₂HPO₄, TES, HEPES, HEPPSO, Triethanolamine, Tricine, Tris, Glycine amide, Bicine, Glycylglycine, TAPS, Boric acid (H₃BO₃/Na₂B₄O₇), CHES, Glycine, NaHCO₃/Na₂CO₃, CAPS, Piperidine, Na₂HPO₄/Na₃PO₄, combinations thereof, etc.

In some embodiments, bead-beating with very high density yttria-stabilized zirconium-oxide beads provides very rapid results and higher quality nucleic acids than those obtain from protocols employing lengthy incubation steps when nucleases have the opportunity to degrade nucleic acids. Bead-beating is a sample homogenization and cell lysis method in which a biological sample (e.g. organism, tissue, cell) is agitated (e.g. vigorously agitated) with beads (e.g. glass or other material) to break up the sample and lyse cells through physical means. Higher density beads allow for shorter bead-beating times and result in overall higher yields than the standard and less dense zirconium-silica beads. The mechanical nature of the lysis by bead beating in conjunction with the enzymatic and chemical lysis results in improved effectiveness against hard to lyse organisms such as Gram-positive bacteria, yeast and spores.

Provided herein are methods, compositions (e.g., reaction mixtures comprising a plurality of PCR primer pairs hybridized to microbial nucleic acids isolated using the methods described herein), kits, and related systems for the isolation, purification, and analysis of total DNA and RNA from a subject or sample. In some embodiments, analysis of DNA and/or RNA isolated and or purified by the present invention comprises amplification and/or mass spectrometry analysis of DNA and/or RNA. In some embodiments, primers are selected to hybridize to conserved sequence regions of nucleic acids derived from a subject or sample (e.g. microorganisms found in blood) and which flank variable sequence regions to yield an identifying amplicon that can be amplified and that is amenable to molecular mass determination. In some embodiments, the molecular mass is converted to a base composition, which indicates the number of each nucleotide in the amplicon. Systems employing software and hardware useful in converting molecular mass data into base composition information are available from, for example, Ibis Biosciences, Inc. (Carlsbad, Calif.), for example the Ibis T5000 Biosensor System, and are described in U.S. patent application Ser. No. 10/754,415, filed Jan. 9, 2004, incorporated by reference herein in its entirety. In some embodiments, the molecular mass or corresponding base composition of one or more different amplicons is queried against a database of molecular masses or base compositions indexed to microorganisms and to the primer pair used to generate the amplicon. A match of the measured base composition to a database entry base composition associates the sample to an indexed microbe in the database. Thus, the identity of the unknown microbe is determined. No prior knowledge of the unknown microbe is necessary to make an identification. In some instances, the measured base composition associates with more than one database entry base composition. Thus, a second/subsequent primer pair is used to generate an amplicon, and its measured base composition is similarly compared to the database to determine its identity in triangulation identification. Furthermore, the methods and other aspects of the invention can be applied to rapid parallel multiplex analyses, the results of which can be employed in a triangulation identification strategy. Thus, in some embodiments, the present invention provides rapid throughput and does not require nucleic acid sequencing or knowledge of the linear sequences of nucleobases of the amplified target sequence for microbe detection and identification.

Particular embodiments of the mass-spectrum based detection methods are described in the following patents, patent applications and scientific publications, all of which are herein incorporated by reference as if fully set forth herein: U.S. Pat. Nos. 7,108,974; 7,217,510; 7,226,739; 7,255,992; 7,312,036; 7,339,051; US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; WO2002/070664; WO2003/001976; WO2003/100035; WO2004/009849; WO2004/052175; WO2004/053076; WO2004/053141; WO2004/053164; WO2004/060278; WO2004/093644; WO 2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; WO2007/118222; Ecker et al., Ibis T5000: a universal biosensor approach for microbiology. Nat Rev Microbiol. 2008 Jun. 3.; Ecker et al., Identification of Acinetobacter species and genotyping of Acinetobacter baumannii by multilocus PCR and mass spectrometry. J Clin Microbiol. 2006 August; 44(8):2921-32.; Ecker et al., Rapid identification and strain-typing of respiratory pathogens for epidemic surveillance. Proc Natl Acad Sci USA. 2005 May 31; 102(22):8012-7. Epub 2005 May 23.; Wortmann et al., Genotypic Evolution of Acinetobacter baumannii Strains in an Outbreak Associated With War Trauma. Infect Control Hosp Epidemiol. 2008 June; 29(6):553-555.; Hannis et al., High-resolution genotyping of Campylobacter species by use of PCR and high-throughput mass spectrometry. J Clin Microbiol. 2008 April; 46(4):1220-5.; Blyn et al., Rapid detection and molecular serotyping of adenovirus by use of PCR followed by electrospray ionization mass spectrometry. J Clin Microbiol. 2008 February; 46(2):644-51.; Eshoo et al., Direct broad-range detection of alphaviruses in mosquito extracts. Virology. 2007 Nov. 25; 368(2):286-95.; Sampath et al., Global surveillance of emerging Influenza virus genotypes by mass spectrometry. PLoS ONE. 2007 May 30; 2(5):e489.; Sampath et al., Rapid identification of emerging infectious agents using PCR and electrospray ionization mass spectrometry. Ann N Y Acad Sci. 2007 April; 1102:109-20.; Hujer et al., Analysis of antibiotic resistance genes in multidrug-resistant Acinetobacter sp. isolates from military and civilian patients treated at the Walter Reed Army Medical Center. Antimicrob Agents Chemother. 2006 December; 50(12):4114-23.; Hall et al., Base composition analysis of human mitochondrial DNA using electrospray ionization mass spectrometry: a novel tool for the identification and differentiation of humans. Anal Biochem. 2005 Sep. 1; 344(1):53-69.; Sampath et al., Rapid identification of emerging pathogens: coronavirus. Emerg Infect Dis. 2005 March; 11(3):373-9., each of which is herein incorporated by reference in its entirety.

In some embodiments, the present disclosure provides systems and methods for diagnosing blood borne infections (e.g., sepsis). In some embodiments, the present disclosure provides quantitative measurement of blood borne microbes. Such information finds use in determining a treatment course of action (e.g., selection of antibiotic or anti-fungal agent) and monitoring treatment (e.g., determining when levels of a microbe have been decreased or eliminated). In some embodiments, the present disclosure provides methods of administering a therapy based on the type or antibiotic resistance of microbes identified using the compositions, systems, and methods described herein. For example, in some embodiments, an antibiotic specific for the organism(s) identified, rather than a broad spectrum antibiotic, is administered. In some embodiments, if an antibiotic resistance gene is identified, an antibiotic known to be effective against such organisms is administered.

### EXAMPLES

Although the disclosure herein refers to certain illustrated embodiments, it is to be understood that these embodiments are presented by way of example and not by way of limitation.

### Example 1

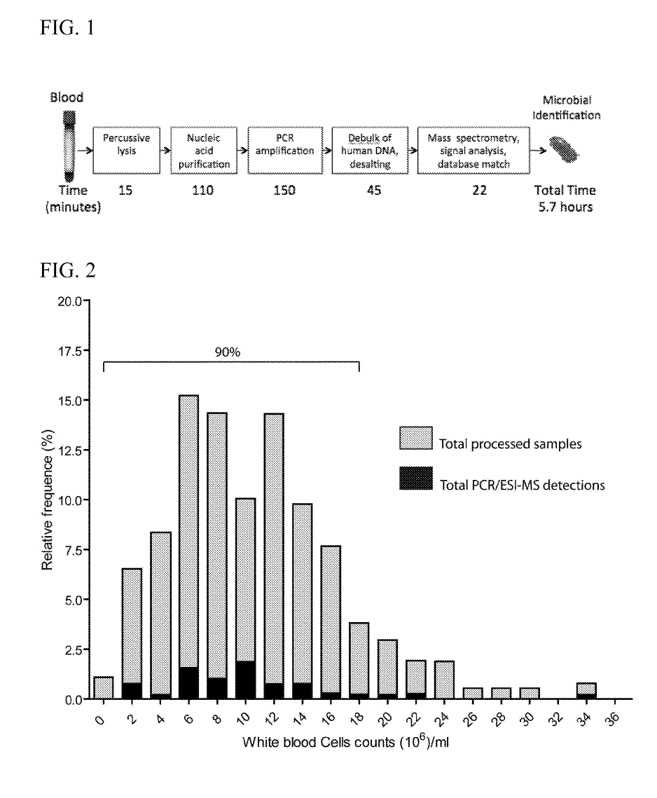

This example describes the performance of an extraction system through analytical limit of detection and breadth of coverage studies using culture-quantified microbes spiked in whole blood. Whole blood specimens from patients with suspected blood stream infections were also evaluated using the system. The PCR/ESI-MS system with high blood volume specimen preparation technology (Ibis Biosciences, Abbott, Carlsbad, Calif.) has sensitivity exceeding that of culture, agrees well with culture data, and is able to detect a broad range of microorganisms rapidly with a work-flow suitable for hospital laboratories. The integrated processes that collectively improve sensitivity and work flow are summarized in FIG. 1.

### Materials and Methods

Extraction and Analysis of DNA from 5 mL of Whole Blood

Genomic DNA was isolated from 5 mL of EDTA-treated whole blood clinical specimens or 5 mL healthy volunteer blood spiked with cultured microbes. Blood samples were lysed in the presence of 665 μl lysis buffer (Abbott Molecular, Des Plaines, Ill., 100 mM Tris solution containing guanidinium thiocyanate and detergent), 145 μl 10% BSA containing a pumpkin DNA extraction control (24), and 3 g of 0.2-mm yttria-stabilized zirconium oxide beads using a large-volume bead mill homogenizer (Ibis Biosciences and Omni International) (speed 6.6 m/s, three 90 sec cycles with 20 sec dwell time). Sample tubes were centrifuged at 3,220 rcf for 5 min. Supernatant fractions were processed by an automated DNA extraction and PCR set-up instrument (Ibis Biosciences, Carlsbad, Calif. and Precision System Science, Co. Ltd, Matsudo, Japan). The extraction system uses pre-filled disposable cartridges containing DNA-free reagents and silica-coated magnetic particles (Abbott Molecular, Des Plaines, Ill.).

Eluates were transferred into 16 wells (30 μl per well) of a custom PCR assay strip pre-filled (25 μl) with 18 unique primer pairs and concentrated PCR master mix. The primers of the Bacteria and Candida assay for Blood Stream Infections (BAC BSI assay) were designed to hybridize to conserved genomic sequences and amplify species-specific genetic signatures from a broad spectrum of bacteria and Candida spp.; target-specific primers yield signatures indicative of antibiotic resistance elements. The gene targets, primer sequences, and configuration have been described in detail previously (28). PCR formulation and thermocycling conditions have also been described elsewhere (25). Due to high loads of white blood cells in 5-mL whole blood specimens, the primer and polymerase concentrations were optimized to enable the BAC BSI assay to withstand potentially extensive interference from high levels of human DNA (up to ˜12 μg per reaction).

### Sequencing and Data Analysis

For identification by sequencing, portions of the 16S rRNA gene (for bacteria) or the 28S rRNA gene (for Candida spp.) were amplified with the primers specified in CLSI guidelines for identification of bacteria and fungi (30), including the primer pairs MM18-A (4F-TTGGAGAGTTTGATCCTGGCTC; SEQ ID NO:1) and 108R (GGCGTGGACTACCAGGGTATCT; SEQ ID NO:2), 28SF (GGACTACCCGCTGAACTTAAGCATATCAATA; SEQ ID NO:3) and 28SR (GGTTTTACACCCAAACACTCGCATAGAC; SEQ ID NO:4), and M13F (CCCAGTCACGACGTTGTAAAACG; SEQ ID NO:5) and M13R (AGCGGATAACAATTTCACACAGG; SEQ ID NO:6), using Platinum Taq High Fidelity (Invitrogen). Platinum Taq buffer was used with 200 μM of each dNTP, 2 mM MgSO₄, and 250 nM of each primer. Reactions were cycled with the following conditions: 95° C. for 2 min; 8 cycles of 95° C. for 15 sec, 52° C. for 45 sec (increasing 0.6° C. per cycle), and 68° C. for 90 sec; 27 cycles of 95° C. for 15 sec, 64° C. for 15 sec, and 68° C. for 60 sec; followed by 4 min at 68° C. SeqWright performed all sequencing.

Sequences analyzed with Phred-Phrap and aligned with BioLign. Primers were trimmed from the alignments and the sequences searched against GenBank using NCBI BLAST. An organism was considered a match to the query sequence if the sequence identity between them was ≧95% along the entire length of the query.

### Clinical Specimens

Blood samples were collected from prospectively consented adults from January, 2012 to April, 2012 at The Johns Hopkins Hospital. Samples were obtained from patients whose physicians ordered blood cultures due to a clinical suspicion of a bloodstream infection. Patients were considered eligible if they were above the age of 18, were having blood cultures drawn as part of clinical care, and were able to provide informed consent. Research specimens were drawn by clinical staff members into 5 mL EDA blood tubes and then shipped at 4° C. for processing.

### Microbiological Methods

Patients suspected of sepsis had two sets of blood cultures obtained after appropriate skin decontamination. A set consisted of one BD BACTEC Plus aerobic/F bottle and one BD BACTEC lytic anaerobic/F bottle (BD Diagnostics). Bottles were sent promptly to the laboratory where they were placed within 1 hr into the BACTEC FX (BD Diagnostics) continuously monitored blood culture system. Bottles were incubated and monitored for five days before being called negative. Positive blood cultures were removed immediately from the instrument and a Gram stain was performed. The peptide nucleic acid fluorescence in situ hybridization (PNA-FISH) Enterococcus faecalis and other Enterococcus dual probe and the PNA-FISH C. albicans/C. glabrata dual probe were used to rapidly identify Gram positive cocci and yeast, respectively. All other pathogens were subcultured to appropriate media depending upon the Gram stain results and the type of bottle from which they were recovered. Organisms were subsequently identified by a variety of phenotypic methods including the Phoenix Automated Microbiology System (BD Diagnostics), classical biochemical analyses, cell wall fatty acid analysis using gas liquid chromatography (Sherlock® Microbial Identification System), and, rarely, 16SrRNA gene sequencing.

### Results

### Optimization of PCR and Desalting Steps for Sensitivity Despite High Levels of Non-Target DNA

Due to very high numbers of white blood cells in 5-ml blood specimens, the BAC assay was optimized in the presence of high levels of human DNA (up to ˜12 μg per reaction). Optimal PCR conditions were determined using systematic matrix analysis that varied primer, Mg⁺⁺, and polymerase concentration, annealing time and temperature. The optimum concentrations for each component were chosen as those that gave the maximum amplicon yields as determined by capillary electrophoresis. Results showed that increasing primer and polymerase concentrations simultaneously to 750 μM and 2.2 units per reaction, respectively, resulted in a PCR yield in a 12-μg DNA background of 86% (56%-120% range) of the yield when 1 μg human DNA was present (data not shown). Varying other parameters resulted in negligible improvements to the previously reported PCR formulations and thermocycling conditions (25).

Prior to mass spectrometry, amplicons are desalted by anion exchange chromatography using an automated platform (31). In addition to salts, which interfere with mass spectrometry, this procedure removes more than 98% of human DNA background in samples containing 12 μg human DNA. During primary amine anion exchange on irregularly shaped porous microparticle clusters, short PCR amplicons bound to sub-micron sized pores on the surface and within the porous cluster. The dual anion exchange and size exclusion properties of the particles prevented the larger-sized human genomic DNA from binding, effectively enriching the sample for PCR amplicons and improving detection by ESI-MS.

### Levels of Human DNA in 5 mL of Blood

In order to define the limit of the human DNA background tolerated, healthy volunteer blood samples were spiked at LOD with multiple microorganisms and analyzed in the presence of a range of human DNA concentrations. The total amount of DNA extracted from 5 ml of blood has been capped to 12 μg per PCR and post-PCR amplicon enrichment reaction. Human DNA from in whole blood samples comes primarily from white blood cells. There is approximately 14 μg of total human DNA in approximately 12×10⁶ cells. The white blood cell counts from the intended use population showed that 90% of patients fell into a range between 0 and 16×10⁶ cells/mL (FIG. 2). Most patients had between 6 and 12×10⁶ white blood cells per mL; 15.1, 14.3, and 14.3% of the subjects had 6, 8, and 12×10⁶ white blood cell/mL, respectively; 90% of patients had 16×10⁶ white blood cells/mL or fewer. To understand the assay capability to withstand higher levels of human DNA, 5-mL whole blood samples were spiked at the assay LOD of 16 CFU/ml of E. faecium (VRE) and 4 CFU/ml of C. albicans with 5, 7, 8, 10, 12, 23, and 40×10⁶ white blood cells/ml. PCR/ESI-MS correctly identified the spiked organisms at all white blood cell levels. These results suggest that microbial DNA will be detected and correctly identified in patients with the highest white blood cells counts observed in this study.

### Limits of Detection for Relevant Organisms

To determine the analytical sensitivity of the PCR/ESI-MS system with the 5-mL blood preparation system, the limits of detection for K. pneumonia (bla_{kpc}+), E. faecium (vanA+/vanB+), S. aureus (mecA⁺), and C. albicans were determined. Each of the primer pairs in the assay target one or more of these organisms. Aliquots of uninfected blood were spiked with a dilution series of microbes in 2-fold steps. The LOD was taken as the last dilution step where of 19 of 20 replicates (95%) were positive. The LODs for each organism (inclusive of their resistance markers) were: S. aureus, 16 CFU/mL; K. pneumonia, 16 CFU/mL; E. faecium, 16 CFU/mL; and C. albicans, 4 CFU/mL. Breadth of coverage of the assay was demonstrated using 70 clinically relevant organisms spiked into 5 mL of EDTA-blood; correct identifications were made for all spiked samples.

### Clinical Specimens

To demonstrate accuracy of organism identification in clinical specimens, 331 prospectively collected, de-identified, consented blood specimens from Johns Hopkins Medical Center emergency department were tested under Institutional Review Board (IRB) approval. PCR/ESI-MS results from analysis of 5 mL of EDTA blood using the BAC Detection Assay were compared with results from standard clinical microbiology cultures. Analysis of 331 patients with suspected bloodstream infections (Table 1) yielded 35 PCR/ESI-MS positive specimens (10.6%) compared to 18 positive specimens by culture (5.4%). Using culture as the comparator method, PCR/ESI-MS was 83% sensitive and 94% specific (Table 2). When PCR/ESI-MS-positive but culture-negative specimens were confirmed by repeat PCR/ESI-MS testing of additional replicate specimens (Table 3), and the confirmed detections were considered true positives, sensitivity increased to 91% and specificity to 99% (see section below). In one specimen two organisms were identify upon culture; both were correctly identified in direct testing by PCR/ESI-MS. PCR/ESI-MS also detected a high level of Candida glabrata in the same sample.

### Sequencing is not an Appropriate Comparator Method for PCR/ESI-MS

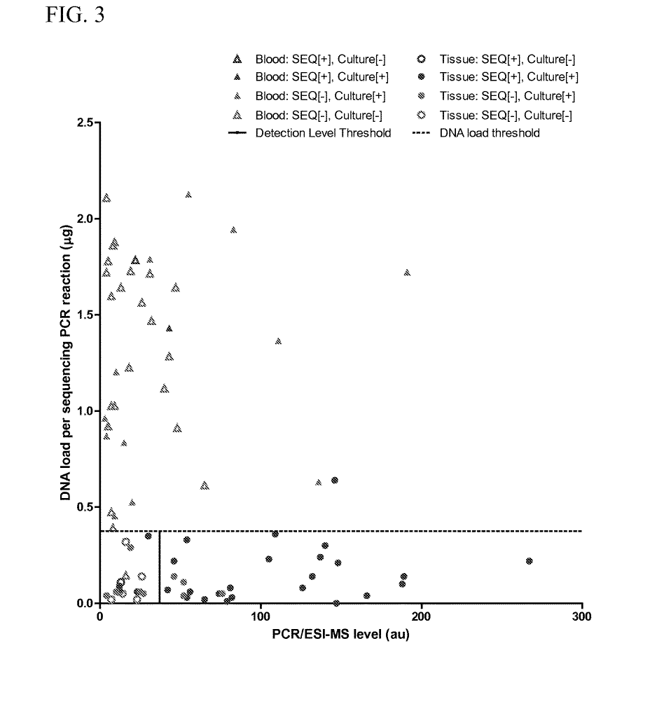

To determine whether sequencing could be used as a comparator method for PCR/ESI-MS, the extracted DNA that was used for PCR/ESI-MS analysis was sequenced using primers and protocols specified by the CLSI guidance document for bacterial and candida identification (32). The specimens from the patient population with suspected bloodstream infections and a second set of samples extracted from orthopedic tissues suspected of being infected (33) were analyzed. The extracted blood specimens had a higher concentration of human DNA (270 ng/μl) than did the tissue specimens (27 ng/μl), but a substantially lower amount of infecting bacterial DNA by PCR/ESI-MS (FIG. 3). Only two of the 35 PCR/ESI-MS-positive specimens (including 15 that were also culture positive) from patients with suspected bloodstream infections could be confirmed by sequencing. Tissue specimens suspected of being infected had a substantially lower amount of human DNA and a higher level of bacterial DNA. Sequencing confirmed that the organisms identified by PCR/ESI-MS in 27 of 36 (75%) tissue specimens were present. No specimens were positive by sequencing and negative by PCR/ESI-MS.

Examination of the data in FIG. 3 indicates that the success of sequencing is dependent on both the concentration of the target bacterial DNA and level of contaminating human DNA. In infected blood specimens, the bacterial DNA load was relatively low and human DNA load was relatively high compared to the infected tissue specimens. Overall, 84% of samples with less than 0.4 μg of background DNA per PCR sequencing reaction and more than 40 genomes per PCR well were positive by sequencing. In contrast, only 8% of the PCR/ESI-MS-positive samples containing over 0.4 μg DNA (all but one of the blood samples tested) were positive by sequencing. Thus, in the absence of a method to remove or reduce human DNA, 16S Sanger sequencing of nucleic acid extracts from whole blood specimens is not a suitable method to identify infecting bacteria or fungi and cannot be used as a comparator method for PCR/ESI-MS of blood specimens.

### Repeated Analytical Testing by PCR/ESI-MS

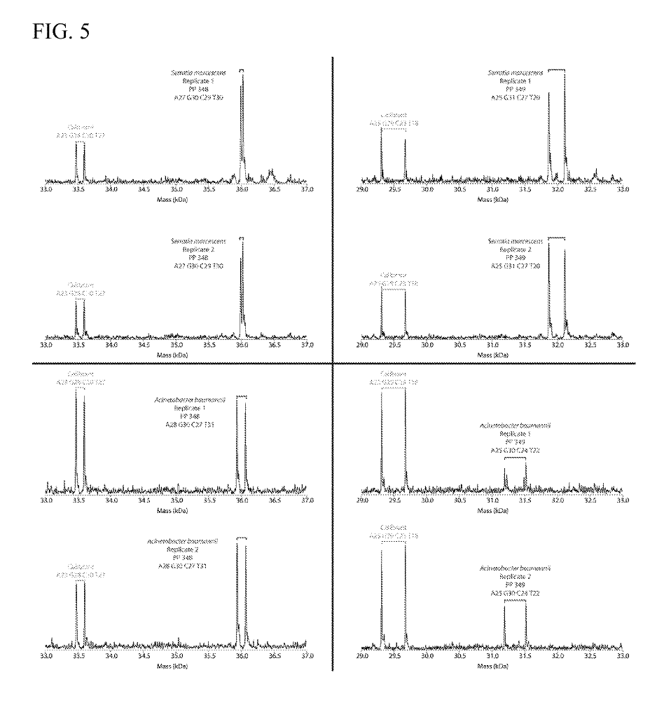

Many studies have shown that molecular methods find bacterial and candida DNA in blood extracts where cultures are negative. Frozen remnant blood samples of the PCR/ESI-MS-positive/culture-negative patient specimens were retested them on a different instrument with a different operator. The results are shown in Table 3. In 18 of 21 cases for which there was sufficient volume of blood to re-test, the results were identical in replicate testing in terms of both the organism identified and the level of microbial DNA detected, providing supporting evidence that the organism DNA reported by PCR/ESI-MS was indeed present in these samples and not the result of post-collection contamination. The three specimens that were negative upon retesting had yielded relatively low-level signals on the initial test. In order to be counted as a detection by PCR/ESI-MS, a critical number of spectra with positive peaks for specific organisms should be achieved. Representative spectra from the specimens that were PCR/ESI-MS-positive in duplicate independent tests are shown in FIG. 5. Species identification results from the completion of a succession of well-defined tasks that are themselves submitted to step-wise stringent quality control requirements strongly indicating that these specimens in fact contain the reported microbial DNA.

Expected Levels of Bacterial DNA in Blood in Patients with Bloodstream Infections

Previously published quantitative microbiology studies have shown that the number of recoverable colony forming units (CFU) of bacteria in the blood of patients with clinically significant bacteremia is low, typically in the range of <1 to 30 CFU/mL (34-36). However, the CFU measured by quantitative microbiology represents only the viable organisms that survive the plating process and does not count dead cells, cells that cannot form colonies, or free microbial DNA that may have been liberated from lysed cells in the blood compartment. Thus, the true concentration of pathogen DNA available in whole blood for molecular analysis in patients with bloodstream infections cannot be inferred from viable cell count (quantitative culture) data.

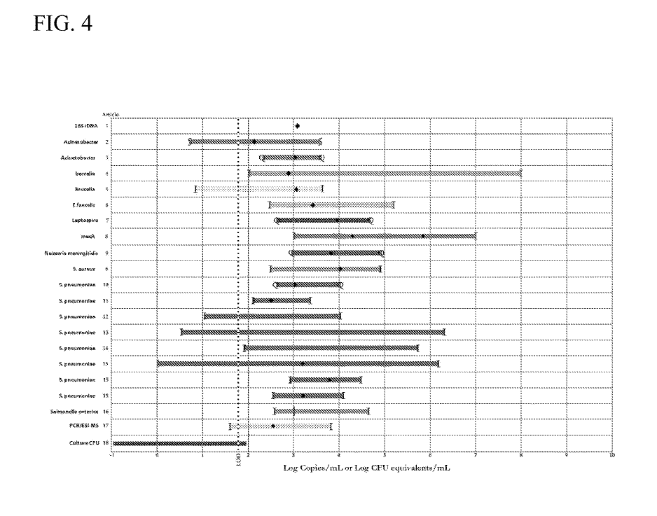

However, organism-specific quantitative PCR has been used to measure of the bacterial DNA present in whole blood specimens from patients with sepsis, pneumonia, or suspected blood stream infections (13, 37-52). Each of these studies analyzed whole blood specimens from patients with suspected or confirmed infections, rather than spiked samples for which the genome to viable cell ratio is expected to be close to 1:1. Of 16 such publications, 15 used a calibrated real-time PCR method focused on single organisms and one publication reported a quantitative 16S broad range method (41). The investigators calibrated their PCR reactions either by using an analytically prepared DNA reference standard of a single copy gene (reporting results as bacterial genome copies/mL) or using quantified spikes of cultured microbes (reporting results as CFU equivalents/mL of blood). Although the results of these experiments revealed some variability between different microorganisms, specific PCR methods and different patient populations, the central values (medians/means) for bacteria were typically between 1×10³ and 1×10⁴ genome copies per mL (FIG. 4). Thus, in patients with suspected or confirmed bloodstream infections the amount of bacterial nucleic acid available for detection by molecular methods is approximately 2-3 orders of magnitude higher than what one might expect from the culture-based quantitative microbiology literature. This explains the apparent inconsistency between the reported analytical LODs of PCR/ESI-MS, which are approximately 16 CFU/mL for freshly spiked blood, the reported concentrations of viable microbial cells in septic blood (averaging between 1 and 10 CFU/ml), and the approximate clinical sensitivity of PCR/ESI-MS of 83-91%. The results reported here are consistent with quantitative PCR analysis of patient specimens (FIG. 4).

An estimate of the bacterial DNA load in patient specimens has clinical utility. Several studies have demonstrated that bacterial load measured by PCR correlates with disease severity and that bacterial load is predictive of which patients are likely to become severely ill (39, 41, 45, 46, 53, 54). Two studies independently identified a bacterial load of 1×10³ genome copies per mL of blood as a “critical threshold” above which patients had increased risk of developing septic shock with E. coli (41), Staphylococcus aureus (41), or Streptococcus pneumonia (46), indicating that quantitative measurement of the bacterial DNA load in blood has clinical value. The analytical sensitivity of PCR/ESI-MS (16 CFU/mL) is more than sufficient to detect the concentrations of organisms present in patients with these serious infections.

TABLE 1
Positive detections by culture or PCR/ESI-MS. The Q score is a single figure of merit
that describes the overall quality of the result derived from primer-dependent parameters
such as the number of primer pairs producing amplification products compared to the maximum
number expected for the identified organism, the closeness of match of those products
to reference signatures in the database for that organism, and consistency of signal amplitudes
across multiple primer pairs. The Q score is a ranking between 0 (lowest) and 1 (highest)
value. The Q score is the output of a Principal Component Analysis and represents a relative
measure of the strength of the data supporting identification. For the BAC BSI assay,
organisms reported as detected have a Q score ≧0.85 in all cases. The Level is a reflection
of signal abundance relative to a set of competitive PCR standards of known input quantity
and thus serves as an indirect estimate of how much specific template was amplified. This
is calculated with reference to an internal calibrant construct (the amplification control)
as described previously (31) and provides a relative measure of the genome (or copy) number
concentration of any detected target.

| | | | | Level |
|---|---|---|---|---|
| Sample # | Culture | PCR/ESI-MS | Q Score | (genomes/well) |
| 1088 | Escherichia coli | Escherichia coli | 0.99 | 55 |
| 1090 | Escherichia coli | Escherichia coli | 0.96 | 3 |
| 1195 | Escherichia coli | Escherichia coli | 0.96 | 10 |
| 1170 | Escherichia coli | Escherichia coli | 0.98 | 136 |
| | Vancomycin Resistant | Enterococcus faecium, | 0.96 | 30 |
| | Enterococcus faecium | vanA | | |
| | Negative | Candida glabrata | 0.97 | 51 |
| 1126 | Klebsiella pneumoniae | Klebsiella pneumoniae | 0.99 | 31 |
| 838 | Klebsiella pneumoniae | Klebsiella pneumoniae | 0.98 | 83 |
| 1414 | Klebsiella pneumoniae | Klebsiella pneumoniae | 0.98 | 4 |
| 1185 | Staphylococcus species, | Staphylococcus epidermidis, | 0.99 | 111 |
| | Coagulase Negative | mecA | | |
| 917 | Staphylococcus species, | Staphylococcus epidermidis, | 0.97 | 10 |
| | Coagulase Negative | mecA | | |
| 1168 | Streptococcus Group G | Streptococcus dysgalactiae | 0.98 | 15 |
| 1230 | Staphylococcus aureus | Staphylococcus aureus | 0.98 | 43 |
| 1296 | Staphylococcus aureus | Staphylococcus aureus | 0.99 | 20 |
| 1366 | Staphylococcus aureus | Staphylococcus aureus | 0.97 | 9 |
| 1407 | Staphylococcus aureus | Staphylococcus aureus | 0.99 | 191 |
| 1346 | Positive, no organism | Escherichia coli | 0.97 | 3 |
| | reported | | | |
| 834 | Viridans Streptococcus | Not Detected | NA | NA |
| | Group | | | |
| 1016 | Escherichia coli | Not Detected | NA | NA |
| 1051 | Streptococcus pneumoniae | Not Detected | NA | NA |
| 933 | Negative | Acinetobacter baumannii | 0.98 | 37 |
| 840 | Negative | Bacteroides fragilis | 0.97 | 18 |
| 869 | Negative | Bartonella henselae | 0.96 | 40 |
| 1093 | Negative | Enterobacter cloacae complex | 0.98 | 19 |
| 1121 | Negative | Enterococcus faecalis | 0.98 | 7 |
| 1381 | Negative | Enterococcus faecalis | 0.98 | 4 |
| 1000 | Negative | Escherichia coli | 0.98 | 73 |
| 1006 | Negative | Escherichia coli | 0.97 | 25 |
| 1023 | Negative | Escherichia coli | 0.95 | 5 |
| 1097 | Negative | Escherichia coli | 0.98 | 48 |
| | Negative | Pseudomonas aeruginosa | 0.98 | 140 |
| 1109 | Negative | Escherichia coli | 0.99 | 8 |
| 1416 | Negative | Escherichia coli | 0.98 | 9 |
| 852 | Negative | Finegoldia magna | 0.90 | 5 |
| 868 | Negative | Finegoldia magna | 0.88 | 4 |
| 885 | Negative | Klebsiella pneumoniae | 0.98 | 18 |
| 800 | Negative | Klebsiella pneumoniae | 0.96 | 8 |
| 1365 | Negative | Klebsiella pneumoniae | 0.99 | 9 |
| 1083 | Negative | Serratia marcescens | 0.99 | 43 |
| 965 | Negative | Staphylococcus aureus | 0.97 | 4 |
| 1181 | Negative | Viridans/Mitis Group | 0.96 | 26 |
| | | Streptococcus | | |

TABLE 2
Concordance of culture results with PCR/ESI-MS on a
per sample basis. Top: Direct comparison of culture
and PCR/ESI-MS. Bottom: Comparison of culture plus
PCR/ESI-MS when replicated as a comparator method.

Culture

| | | positive | negative | Total |
|---|---|---|---|---|
| PCR/ESI-MS | positive | 15 | 20 | 35 |
| | negative | 3 | 293 | 296 |
| | Total | 18 | 313 | 331 |
| | | Sensitivity | 83% | |
| | | Specificity | 94% | |

| | | Culture + Repeated PCR/ESI-MS | | |
|---|---|---|---|---|
| | | positive | negative | Total |
| PCR/ESI-MS | positive | 32 | 3 | 35 |
| | negative | 3 | 293 | 296 |
| | Total | 35 | 296 | 331 |
| | | Sensitivity | 91% | |
| | | Specificity | 99% | |

TABLE 3
Replicate testing of PCR/ESI-MS positive samples that were negative by culture.

| | | Level | | | Level |
|---|---|---|---|---|---|
| PCR/ESI-MS Replicate 1 | Q Score | Genomes/Well | PCR/ESI-MS Replicate 2 | Q Score | Genomes/Well |
| Acinetobacter baumannii | 0.98 | 37 | Acinetobacter baumannii | 0.98 | 22 |
| Bacteroides fragilis | 0.97 | 18 | Bacteroides fragilis | 0.9 | 4 |
| Bartonella henselae | 0.96 | 40 | Bartonella henselae | 0.98 | 32 |
| Candida glabrata | 0.97 | 51 | Candida glabrata | 0.97 | 69 |
| Enterobacter cloacae | 0.98 | 19 | Enterobacter cloacae | 0.98 | 22 |
| complex | | | complex | | |
| Enterococcus faecalis | 0.98 | 7 | No Result | NA | |
| Enterococcus faecalis | 0.98 | 4 | No Result | NA | |
| Escherichia coli | 0.98 | 73 | Escherichia coli | 0.99 | 59 |
| Escherichia coli | 0.97 | 25 | Escherichia coli | 0.99 | 27 |
| Escherichia coli | 0.95 | 5 | Escherichia coli | 0.98 | 6 |
| Escherichia coli | 0.98 | 48 | Escherichia coli | 0.98 | 43 |
| Escherichia coli | 0.99 | 8 | Escherichia coli | 0.98 | 10 |
| Escherichia coli | 0.98 | 9 | Escherichia coli | 0.96 | 8 |
| Finegoldia magna | 0.90 | 5 | Finegoldia magna | 0.99 | 8 |
| Finegoldia magna | 0.88 | 4 | Finegoldia magna | 0.98 | 6 |
| Klebsiella pneumoniae | 0.98 | 18 | Klebsiella pneumoniae | 0.98 | 23 |
| Klebsiella pneumoniae | 0.96 | 8 | No Result | NA | |
| Klebsiella pneumoniae | 0.99 | 9 | Klebsiella pneumoniae | 0.98 | 10 |
| Pseudomonas aeruginosa | 0.98 | 140 | Pseudomonas aeruginosa | 0.99 | 164 |
| Serratia marcescens | 0.99 | 43 | Serratia marcescens | 0.98 | 41 |
| Staphylococcus aureus | 0.97 | 4 | Staphylococcus aureus | 0.99 | 8 |

### REFERENCES

1. Kumar A, Roberts D, Wood K E, Light B, Parrillo J E, Sharma S, Suppes R, Feinstein D, Zanotti S, Taiberg L, Gurka D, Kumar A, Cheang M. 2006. Duration of hypotension before initiation of effective antimicrobial therapy is the critical determinant of survival in human septic shock. Crit Care Med 34:1589-1596.
2. Dellinger R P, Levy M M, Rhodes A, Annane D, Gerlach H, Opal S M, Sevransky J E, Sprung C L, Douglas I S, Jaeschke R, Osborn T M, Nunnally M E, Townsend S R, Reinhart K, Kleinpell R M, Angus D C, Deutschman C S, Machado F R, Rubenfeld G D, Webb S A, Beale R J, Vincent J L, Moreno R. 2013. Surviving sepsis campaign: international guidelines for management of severe sepsis and septic shock: 2012. Critical Care Medicine 41:580-637.
3. Dellinger R P, Levy M M, Carlet J M, Bion J, Parker M M, Jaeschke R, Reinhart K, Angus D C, Brun-Buisson C, Beale R, Calandra T, Dhainaut J F, Gerlach H, Harvey M, Marini J J, Marshall J, Ranieri M, Ramsay G, Sevransky J, Thompson B T, Townsend S, Vender J S, Zimmerman J L, Vincent J L. 2008. Surviving Sepsis Campaign: international guidelines for management of severe sepsis and septic shock: 2008. Crit Care Med 36:296-327.
4. Kuti E L, Patel A A, Coleman C I. 2008. Impact of inappropriate antibiotic therapy on mortality in patients with ventilator-associated pneumonia and blood stream infection: a meta-analysis. J Crit Care 23:91-100.
5. Kollef M H. 2008. Broad-spectrum antimicrobials and the treatment of serious bacterial infections: getting it right up front. Clin Infect Dis 47 Suppl 1:S3-13.
6. Fenollar F, Raoult D. 2007. Molecular diagnosis of bloodstream infections caused by non-cultivable bacteria. Int J Antimicrob Agents 30 Suppl 1:S7-15.
7. Rangel-Frausto M S, Pittet D, Costigan M, Hwang T, Davis C S, Wenzel R P. 1995. The natural history of the systemic inflammatory response syndrome (SIRS). A prospective study. Jama 273:117-123.
8. Lamy B, Roy P, Carret G, Flandrois J-P, Delignette-Muller M L. 2002. What is the relevance of obtaining multiple blood samples for culture? A comprehensive model to optimize the strategy for diagnosing bacteremia. CID 35:842-850.
9. Reimer L G, Wilson M L, Weinstein M P. 1997. Update on detection of bacteremia and fungemia. Clin Microbiol Rev 10:444-465.
10. Weinstein M P. 1996. Current blood culture methods and systems: clinical concepts, technology, and interpretation of results. Clin Infect Dis 23:40-46.
11. Morata P, Queipo-Ortuno M I, de Dios Colmenero J. 1998. Strategy for optimizing DNA amplification in a peripheral blood PCR assay used for diagnosis of human brucellosis. J Clin Microbiol 36:2443-2446.
12. Cogswell F B, Bantar C E, Hughes T G, Gu Y, Philipp M T. 1996. Host DNA can interfere with detection of Borrelia burgdorferi in skin biopsy specimens by PCR. J Clin Microbiol 34:980-982.
13. Peters R P, van Agtmael M A, Gierveld S, Danner S A, Groeneveld A B, Vandenbroucke-Grauls C M, Savelkoul P H. 2007. Quantitative detection of Staphylococcus aureus and Enterococcus faecalis DNA in blood to diagnose bacteremia in patients in the intensive care unit. J Clin Microbiol 45:3641-3646.
14. Handschur M, Karlic H, Hertel C, Pfeilstocker M, Haslberger A G. 2009. Preanalytic removal of human DNA eliminates false signals in general 16S rDNA PCR monitoring of bacterial pathogens in blood. Comp Immunol Microbiol Infect Dis 32:207-219.
15. Gebert S, Siegel D, Wellinghausen N. 2008. Rapid detection of pathogens in blood culture bottles by real-time PCR in conjunction with the pre-analytic tool MolYsis. J Infect 57:307-316.
16. Hansen W L, Bruggeman C A, Wolffs P F. 2009. Evaluation of new preanalysis sample treatment tools and DNA isolation protocols to improve bacterial pathogen detection in whole blood. J Clin Microbiol 47:2629-2631.
17. Wellinghausen N, Kochem A J, Disque C, Muhl H, Gebert S, Winter J, Matten J, Sakka S G. 2009. Diagnosis of bacteremia in whole-blood samples by use of a commercial universal 16S rRNA gene-based PCR and sequence analysis. J Clin Microbiol 47:2759-2765.
18. Sachse S, Straube E, Lehmann M, Bauer M, Russwurm S, Schmidt K H. 2009. Truncated human cytidylate-phosphate-deoxyguanylate-binding protein for improved nucleic acid amplification technique-based detection of bacterial species in human samples. J Clin Microbiol 47:1050-1057.
19. Lehmann L E, Alvarez J, Hunfeld K P, Goglio A, Kost G J, Louie R F, Raglio A, Regueiro B J, Wissing H, Stuber F. 2009. Potential clinical utility of polymerase chain reaction in microbiological testing for sepsis. Crit Care Med 37:3085-3090.
20. Schreiber J, Nierhaus A, Braune S A, de Heer G, Kluge S. 2013. Comparison of three different commercial PCR assays for the detection of pathogens in critically ill sepsis patients. Med Klin Intensivmed Notfmed 108:311-318.
21. Ecker D J, Sampath R, Li H, Massire C, Matthews H E, Toleno D, Hall T A, Blyn L B, Eshoo M W, Ranken R, Hofstadler S A, Tang Y W. 2010. New technology for rapid molecular diagnosis of bloodstream infections. Expert Rev Mol Diagn 10:399-415.
22. Kaleta E J, Clark A E, Cherkaoui A, Wysocki V H, Ingram E L, Schrenzel J, Wolk D M. 2011. Comparative analysis of PCR-electrospray ionization/mass spectrometry (M S) and MALDI-TOF/M S for the identification of bacteria and yeast from positive blood culture bottles. Clin Chem 57:1057-1067.
23. Kaleta E J, Clark A E, Johnson D R, Gamage D C, Wysocki V H, Cherkaoui A, Schrenzel J, Wolk D M. 2011. Use of PCR coupled with electrospray ionization mass spectrometry for rapid identification of bacterial and yeast bloodstream pathogens from blood culture bottles. J Clin Microbiol 49:345-353.
24. Metzgar D, Frinder M, Lovari R, Toleno D, Massire C, Blyn L B, Ranken R, Carolan H E, Hall T A, Moore D, Hansen C J, Sampath R, Ecker D J. 2013. Broad-spectrum biosensor capable of detecting and identifying diverse bacterial and Candida species in blood. J. Clin. Microbiol. 51:2670-2678.
25. Eshoo M W, Crowder C D, Li H, Matthews H E, Meng S, Sefers S E, Sampath R, Stratton C W, Blyn L B, Ecker D J, Tang Y W. 2010. Detection and identification of Ehrlichia species in blood by use of PCR and electrospray ionization mass spectrometry. J Clin Microbiol 48:472-478.
26. Crowder C D, Matthews H, Schutzer S, Rounds M A, Luft B J, Nolte O, Campbell S R, Phillipson C A, Li F, Sampath R, Ecker D J, Eshoo M W. 2010. Genotypic variation and mixtures of Lyme Borrelia in Ixodes ticks from North America and Europe. PLoS ONE 5:E10650 10651-10659.
27. Farrell J J, Sampath R, Bonomo R A. 2011. Identifying Pathogens in “Culture Negative” Infections: a Case Series exploring PCR and Electrospray Ionization Mass Spectrometry for Microbial Identification, IDSA Poster, Boston, Mass.
28. Laffler T G, Cummins L L, McClain C M, Quinn C D, Toro M A, Carolan H E, Toleno D M, Rounds M A, Eshoo M, Stratton C W, Sampath R, Blyn L B, Ecker D J, Tang Y W. 2013. Enhanced Diagnostic Yields of Bacteremia and Candidemia in Blood Specimens by PCR/Electrospray Ionization Mass Spectrometry. Journal of Clinical Microbiology 10.1128/JCM.00876-13.
29. Jordana-Lluch E, Carolan H E, Gimenez M, Sampath R, Ecker D J, Quesada M D, Modol J M, Armestar F, Blyn L B, Cummins L L, Ausina V, Martro E. 2013. Rapid diagnosis of bloodstream infections with PCR followed by mass spectrometry. PLoS One 8:e62108.
30. Petti C A, Bosshard P P, Brandt M E, Clarridge J E, Feldblyum T V, Foxall P, Furtado M R, Pace N, Procop G. 2008. Interpretive Criteria for Identification of Bacteria and Fungi by DNA Target Sequencing; Approved Guideline. Clinical and Laboratory Standards Institute 28:88.
31. Hofstadler S A, Sampath R, Blyn L B, Eshoo M W, Hall T A, Jiang Y, Drader J J, Hannis J C, Sannes-Lowery K A, Cummins L L, Libby B, Walcott D J, Schink A, Massire C, Ranken R, White N, Samant V, McNeil J A, Knize D, Robbins D, Rudnik K, Desai A, Moradi E, Ecker D J. 2005. TIGER: the universal biosensor. Inter. J. Mass Spectrom. 242:23-41.
32. Petti C A, Bosshard P P, Brandt M E, Clarridge J E, Feldblyum T V, Foxall P, Furtado M R, Pace N, Procop G. 2008. Interpretive Criteria for Identification of Bacteria and Fungi by DNA Target Sequencing; Approved Guideline, Document MM18-A. Clinical and Laboratory Standards Institute 28:1-73.
33. Bowers D R, Hunter A S, Jacobs D M, Kuper K M, Musick W L, Perez K K, Shah D N, Schilling A N. 2013. Significant publications on infectious diseases pharmacotherapy in 2012. American journal of health-system pharmacy: AJHP: official journal of the American Society of Health-System Pharmacists 70:1930-1940.
34. Werner A S, Cobbs C G, Kaye D, Hook E W. 1967. Studies on the bacteremia of bacterial endocarditis. Jama 202:199-203.
35. Kreger B E, Craven D E, Carling P C, McCabe W R. 1980. Gram-negative bacteremia. III. Reassessment of etiology, epidemiology and ecology in 612 patients.

Am J Med 68:332-343.
36. Henry N K, McLimans C A, Wright A J, Thompson R L, Wilson W R, Washington J A, 2nd. 1983. Microbiological and clinical evaluation of the isolator lysis-centrifugation blood culture tube. J Clin Microbiol 17:864-869.
37. Chuang Y C, Chang S C, Wang W K. 2012. Using the rate of bacterial clearance determined by real-time polymerase chain reaction as a timely surrogate marker to evaluate the appropriateness of antibiotic usage in critical patients with Acinetobacter baumannii bacteremia. Crit Care Med 40:2273-2280.
38. Agampodi S B, Matthias M A, Moreno A C, Vinetz J M. 2012. Utility of quantitative polymerase chain reaction in leptospirosis diagnosis: association of level of leptospiremia and clinical manifestations in Sri Lanka. Clin Infect Dis 54:1249-1255.
39. Darton T, Guiver M, Naylor S, Jack D L, Kaczmarski E B, Borrow R, Read R C. 2009. Severity of meningococcal disease associated with genomic bacterial load. Clin Infect Dis 48:587-594.
40. Massi M N, Shirakawa T, Gotoh A, Bishnu A, Hatta M, Kawabata M. 2005. Quantitative detection of Salmonella enterica serovar Typhi from blood of suspected typhoid fever patients by real-time PCR. Int J Med Microbiol 295:117-120.
41. Kirkbright S, Fatovich D, Kee C, Kay I, Flexman J, Pryce T M, Waterer G W. 2011. Quantitative rt-PCR holds promise as a screening tool for patients with severe sepsis. Emerg Med Australas 23:502-506.
42. Reier-Nilsen T, Farstad T, Nakstad B, Lauvrak V, Steinbakk M. 2009. Comparison of broad range 16S rDNA PCR and conventional blood culture for diagnosis of sepsis in the newborn: a case control study. BMC Pediatr 9:5.
43. Chuang Y C, Chang S C, Wang W K. 2010. High and increasing Oxa-51 DNA load predict mortality in Acinetobacter baumannii bacteremia: implication for pathogenesis and evaluation of therapy. PLoS One 5:e14133.
44. Reller M E, Clemens E G, Schachterle S E, Mtove G A, Sullivan D J, Dumler J S. 2011. Multiplex 5′ nuclease-quantitative PCR for diagnosis of relapsing fever in a large Tanzanian cohort. J Clin Microbiol 49:3245-3249.
45. Ho Y C, Chang S C, Lin S R, Wang W K. 2009. High levels of mecA DNA detected by a quantitative real-time PCR assay are associated with mortality in patients with methicillin-resistant Staphylococcus aureus bacteremia. J Clin Microbiol 47:1443-1451.
46. Rello J, Lisboa T, Lujan M, Gallego M, Kee C, Kay I, Lopez D, Waterer G W. 2009. Severity of pneumococcal pneumonia associated with genomic bacterial load. Chest 136:832-840.
47. Navarro E, Segura J C, Castano M J, Solera J. 2006. Use of real-time quantitative polymerase chain reaction to monitor the evolution of Brucella melitensis DNA load during therapy and post-therapy follow-up in patients with brucellosis. Clin Infect Dis 42:1266-1273.
48. Peters R P, de Boer R F, Schuurman T, Gierveld S, Kooistra-Smid M, van Agtmael M A, Vandenbroucke-Grauls C M, Persoons M C, Savelkoul P H. 2009. Streptococcus pneumoniae DNA load in blood as a marker of infection in patients with community-acquired pneumonia. J Clin Microbiol 47:3308-3312.
49. Kee C, Palladino S, Kay I, Pryce T M, Murray R, Rello J, Gallego M, Lujan M, Munoz-Almagro C, Waterer G W. 2008. Feasibility of real-time polymerase chain reaction in whole blood to identify Streptococcus pneumoniae in patients with community-acquired pneumonia. Diagn Microbiol Infect Dis 61:72-75.
50. Kee C, Fatovich D M, Palladino S, Kay I D, Pryce T M, Flexman J, Murray R, Waterer G W. 2010. Specificity of a quantitative real-time polymerase chain reaction assay for the detection of invasive pneumococcal disease: identifying streptococcus pneumoniae using quantitative polymerase chain reaction. Chest 137:243-244.
51. van Haeften R, Palladino S, Kay I, Keil T, Heath C, Waterer G W. 2003. A quantitative LightCycler PCR to detect Streptococcus pneumoniae in blood and CSF. Diagn Microbiology and Infectious Disease 47:407-414.
52. Carrol E D, Guiver M, Nkhoma S, Mankhambo L A, Marsh J, Balmer P, Banda D L, Jeffers G, White S A, Molyneux E M, Molyneux M E, Smyth R L, Hart C A. 2007. High pneumococcal DNA loads are associated with mortality in Malawian children with invasive pneumococcal disease. The Pediatric Infectious Disease Journal 26:416-422.
53. Waterer G, Rello J. 2011. Why should we measure bacterial load when treating community-acquired pneumonia? Curr Opin Infect Dis 24:137-141.
54. Ovstebo R, Brandtzaeg P, Brusletto B, Haug K B, Lande K, Hoiby E A, Kierulf P. 2004. Use of robotized DNA isolation and real-time PCR to quantify and identify close correlation between levels of Neisseria meningitidis DNA and lipopolysaccharides in plasma and cerebrospinal fluid from patients with systemic meningococcal disease. J Clin Microbiol 42:2980-2987.
55. Perez K K, Olsen R J, Musick W L, Cernoch P L, Davis J R, Land G A, Peterson L E, Musser J M. 2013. Integrating rapid pathogen identification and antimicrobial stewardship significantly decreases hospital costs. Archives of pathology & laboratory medicine 137:1247-1254.
56. Huang A M, Newton D, Kunapuli A, Gandhi T N, Washer L L, Isip J, Collins C D, Nagel J L. 2013. Impact of rapid organism identification via matrix-assisted laser desorption/ionization time-of-flight combined with antimicrobial stewardship team intervention in adult patients with bacteremia and candidemia. Clinical infectious diseases: an official publication of the Infectious Diseases Society of America 57:1237-1245.
57. Davies J, Gordon C L, Tong S Y, Baird R W, Davis J S. 2012. Impact of results of a rapid Staphylococcus aureus diagnostic test on prescribing of antibiotics for patients with clustered gram-positive cocci in blood cultures. J. Clin. Microbiol. 50:2056-2058.
58. Sango A, McCarter Y S, Johnson D, Ferreira J, Guzman N, Jankowski C A. 2013. Stewardship approach for optimizing antimicrobial therapy through use of a rapid microarray assay on blood cultures positive for Enterococcus species. J Clin Microbiol 51:4008-4011.
59. Farrell J J, Sampath R, Ecker D, Bonomo R A. 2013. Salvage Microbiology: Detection of Bacteria Directly from Clinical Specimens following Initiation of antimicrobial Treatment. PLoS One 8.
60. Farrell J J, Tsung A J, Flier L, Martinez D L, Beam S B, Chen C, Lowery K S, Sampath R, Bonomo R A. 2013. PCR and Electrospray Ionization Mass Spectrometry (PCR/ESI-M S): Detection of Persistent Enterococcus faecalis in Cerebrospinal Fluid following Treatment of Post-Operative Ventriculitis. J. Clin. Microbiol.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method of detecting a microbe in a blood sample, comprising
a) lysing a whole blood sample from a subject in a lysis buffer comprising yttria-stabilized zirconium oxide beads using a large-volume bead mill homogenizer;
b) processing the supernatant fractions from said lysis using an automated DNA extraction system that uses pre-filled disposable cartridges containing DNA-free reagents and silica-coated magnetic particles; and
c) performing a PCR reaction on the eluate of said processing using a plurality of PCR primer pairs, wherein each of said PCR primer pairs hybridizes to a conserved genomic sequence of a microbe.

2. The method of claim 1, further comprising the step of performing electrospray ionization mass spectrometry (ESI-MS) on amplicons of said PCR reaction, wherein said ESI-MS determines the presence or absence of said microbe in said sample.

3. The method of claim 1, wherein PCR reaction utilizes primer pairs at a concentration of 750 μM and polymerase at a concentration of 2.2 units per reaction.

4. The method of claim 1, wherein said PCR reaction further comprises a plurality of target specific primers that hybridize to antibiotic resistant elements of microbial DNA.

5. The method of claim 1, wherein said microbes are selected from the group consisting of bacteria and yeast.

6. The method of claim 5, wherein said bacteria are selected from the group consisting of K. pneumonia, E. faecium, and S. aureus.

7. The method of claim 5, wherein said yeast is C. albicans.

8. The method of claim 6, wherein said bacteria comprise one or more antibiotic resistance genes.

9. The method of claim 1, wherein said PCR reaction is configured to amplify microbial DNA in a sample comprising up to 12 μg of human DNA per reaction.

10. The method of claim 1, wherein said lysis buffer comprises 3 g of 0.2-mm yttria-stabilized zirconium oxide beads.

11. The method of claim 1, wherein said method detects said microbe with a limit of detection of 20 CFU/ML or less.

12. The method of claim 1, wherein said method detects said microbe with a limit of detection of 16 CFU/ML or less.

13. The method of claim 1, wherein said method detects said microbe with a limit of detection of 10 CFU/ML or less.

14. The method of claim 1, wherein said method detects said microbe with a limit of detection of 4 CFU/ML or less.

15. The method of claim 2, wherein the presence of said microbe in said sample is indicative of a diagnosis of sepsis, septic shock, pneumonia, or a blood stream infection in said subject.

16. The method of claim 15, further comprising the step of determining a treatment course of action based on said diagnosis.

17. The method of claim 16, wherein said treatment course of action comprises administration of an antibiotic or anti-fungal agent to said subject.

18. The method of claim 2, wherein said ESI-MS further determines the identity of said microbes and/or the presence of absence of antibiotic resistance genes in said microbes.

19. The method of claim 16, wherein said antibiotic is specific for said microbe or targets antibiotic resistance microbes.

20. The method of claim 16, further comprising the step of administering said treatment.

21. The method of claim 1, wherein said blood sample is approximately 5 ml.

22. The use of the method of claim 1 to diagnose or monitor sepsis, septic shock, pneumonia, or a blood stream infection in said subject.

23. A kit, comprising one or more reagents useful, necessary, or sufficient for detecting a microbe in a blood sample selected from the group consisting of a lysis buffer comprising yttria-stabilized zirconium oxide beads, a plurality of PCR primer pairs, wherein each of the PCR primer pairs hybridizes to a conserved genomic sequence of a microbe, and a DNA polymerase.

24. The kit of claim 23, further comprising a plurality of target specific primers that hybridize to antibiotic resistant elements of microbial DNA.

25. A system, comprising:
a) the kit of claim 23;
b) a large-volume bead mill homogenizer; and
c) an automated DNA extraction system that uses pre-filled disposable cartridges containing DNA-free reagents and silica-coated magnetic particles); and optionally an ESI-MS instrument.

26. (canceled)
